(19) United States
(12) Patent Application Publication
(10) Pub. No.: US 2019/0133493 A1
(43) Pub. Date: May 9, 2019

(54) METHOD AND SYSTEM FOR ANALYZING GAIT

(71) Applicant: NATIONAL CHENG KUNG UNIVERSITY, Tainan City (TW)

(72) Inventors: Wei-Chih LIEN, Taipei City (TW); Wen-Fong WANG, Kaohsiung City (TW); Ta-Shen KUAN, Tainan City (TW); Pei-Fang SU, Tainan City (TW); Yung-Heng CHENG, Yunlin County (TW); Yu-Lun ZHENG, Tainan City (TW); Chao-Hsien HSIEH, Tainan City (TW)

(21) Appl. No.: 15/826,629

(22) Filed: Nov. 29, 2017

(30) Foreign Application Priority Data
Nov. 3, 2017 (TW) 106138187

(51) Int. Cl. A61B 5/11; A61B 5/00; G06F 17/11

(52) U.S. Cl. CPC A61B 5/112; A61B 5/7246; A61B 5/7267; G06F 17/11; A61B 2562/0219; A61B 5/725; A61B 5/7257; A61B 5/7465; A61B 5/7435

(57) ABSTRACT

A method for analyzing gait is provided for a system including multiple accelerometers. The method includes: for each time point and each accelerometer, calculating a root mean square (RMS) value according to the accelerations sensed on sensing axes of the corresponding accelerometer; calculating a cross correlation coefficient according to the RMS values of a first accelerometer and a second accelerometer; calculating a first auto-correlation coefficient of the RMS values of the first accelerometer; calculating a second auto-correlation coefficient of the RMS values of the second accelerometer; and calculating a first gait index according to the cross correlation coefficient, the first auto-correlation coefficient, and the second auto-correlation coefficient.

### RELATED APPLICATIONS

This application claims priority to Taiwan Application Serial Number 106138187 filed Nov. 3, 2017, which is herein incorporated by reference.

### BACKGROUND

### Field of Invention

The present invention relates to a gait analyzing method. More particularly, the present invention relates to a method and a system for analyzing gait by using accelerometers.

### Description of Related Art

Asymmetric gait impairs walking performance and even leads to falls. Gait analysis provides a doctor with information that the doctor can make appropriate treatment plans. For example, the information of the gait analysis may be used for determining whether an ankle or a knee is abnormal, and therefore an appropriate treatment can be provided for the right location. On the other hand, falls are common among the elderly or chronic stroke patients. Falls can have serious consequence in this population. For example, individuals with stroke are much more likely to sustain a hip fracture due to a fall and to lose independent mobility. Therefore, it is a technical issue for the people in the art about how the gait analysis is performed effectively.

### SUMMARY

Embodiments of the invention provide a gait analyzing method for a gait analyzing system including multiple accelerometers. Each of the accelerometers has multiple sensing axes. The gait analyzing method includes: calculating a root mean square (RMS) value for each of multiple time points and each of the accelerometers according to multiple accelerations sensed on the sensing axes of the corresponding accelerometer, in which the accelerometers includes a first accelerometer and a second accelerometer, the first accelerometer corresponds to a first lower extremity of a gait, the second accelerometer corresponds to a second lower extremity of the gait, and the first lower extremity is different from the second lower extremity; calculating a cross correlation coefficient according to the RMS values of the first accelerometer and the second accelerometer; calculating a first auto-correlation coefficient of the RMS values of the first accelerometer; calculating a second auto-correlation coefficient of the RMS values of the second accelerometer; and calculating a first gait index associated to the gait according to the cross correlation coefficient, the first auto-correlation coefficient, and the second auto-correlation coefficient.

In some embodiments, the step of calculating the cross correlation coefficient is performed according to the following equation (1).

Cc(k)=Σₙ₌₁^{N}a1₍ₙ₎a2₍ₙ₋ₖ₎ k=0,±1,±2, . . . , ±N−1 (1)
if n−k≤0 or n−k≥N, then a2₍ₙ₋ₖ₎=0
   k represents one of the time points, N represents the number of the time points, Cc(k) represents the cross correlation coefficient at the time point k, a1₍ₙ₎ represents the RMS value of the first accelerometer at a time point n, a2₍ₙ₋ₖ₎ represents the RMS value of the second accelerometer at a time point (n−k).

In some embodiments, the step of calculating the first auto-correlation coefficient is performed according to the following equation (2).

Ac1₍ₖ₎=Σₙ₌₁^{N}a1₍ₙ₎a1₍ₙ₋ₖ₎ k=0,±1,±2, . . . , ±N−1 (2)
if n−k≤0 or n−k≥N, then a1₍ₙ₋ₖ₎=0
   The step of calculating the second auto-correlation coefficient is performed according to the following equation (3).

Ac2₍ₖ₎=Σₙ₌₁^{N}a2₍ₙ₎a2₍ₙ₋ₖ₎ k=0,±1,±2, . . . , ±N−1 (3)
if n−k≤0 or n−k≥N, then a2₍ₙ₋ₖ₎=0

In some embodiments, the step of calculating the first gait index of the gait is performed according to the following equation (4).

$${Cc}_{norm} = \frac{max ( Cc )}{\sqrt{A c {1}_{( 0 )} \times Ac {2}_{( 0 )}}} \qquad ( 4 )$$

In some embodiments, the gait analyzing method further includes: calculating a delay time that the cross correlation coefficient reaches a maximum value; and obtaining a second gait index by normalizing the delay time according to the number of the time points.

In some embodiments, the gait analyzing method further includes: training a machine learning model according to the first gait index and the second gait index, determining whether the gait is normal according to the machine learning model.

In some embodiments, the gait analyzing method further includes: performing a recurrence quantification analysis on the RMS values of one of the accelerometers, and displaying a recurrence plot on a screen.

From another aspect, embodiments of the invention provide a gait analyzing system including multiple accelerometer and a controller. Each of the accelerometers has multiple sensing axes. The accelerometers includes a first accelerometer and a second accelerometer. The first accelerometer corresponds to a first lower extremity of a gait, the second accelerometer corresponds to a second lower extremity of the gait, and the first lower extremity is different from the second lower extremity. The controller is configured to receive multiple acceleration sensed on the sensing axes of each of the accelerometers. The controller calculates a root mean square (RMS) value for each of multiple time points and each of the accelerometers according to the accelerations sensed on the sensing axes of the corresponding accelerometer, calculates a cross correlation coefficient according to the RMS values of the first accelerometer and the second accelerometer, calculates a first auto-correlation coefficient of the RMS values of the first accelerometer, calculates a second auto-correlation coefficient of the RMS values of the second accelerometer, and calculates a first gait index associated to the gait according to the cross correlation coefficient, the first auto-correlation coefficient, and the second auto-correlation coefficient.

In some embodiments, the controller calculates the cross correlation coefficient according to the equation (1). In some embodiments, the controller calculates the first auto-correlation coefficient according to the equation (2). The controller calculates the second auto-correlation coefficient according to the equation (3). In some embodiments, the controller calculates the first gait index according to the equation (4).

In some embodiments, the controller calculates a delay time that the cross correlation coefficient reaches a maximum value, and obtains a second gait index by normalizing the delay time according to the number of the time points.

### BRIEF DESCRIPTION OF THE DRAWINGS

The invention can be more fully understood by reading the following detailed description of the embodiment, with reference made to the accompanying drawings as follows.

FIG. 1 is a schematic diagram of a gait analyzing system in accordance with an embodiment.

FIG. 2 is a schematic diagram of dividing gaits in accordance with an embodiment.

FIG. 3 is a schematic diagram of calculating a second gait index in accordance with an embodiment.

FIG. 4 is a schematic diagram of a graphical interface in accordance with an embodiment.

FIG. 5 shows a flow chart of a gait analyzing method in accordance with an embodiment.

FIG. 6 is a diagram illustrating SVM classification in accordance with an embodiment.

### DETAILED DESCRIPTION

Specific embodiments of the present invention are further described in detail below with reference to the accompanying drawings, however, the embodiments described are not intended to limit the present invention and it is not intended for the description of operation to limit the order of implementation. Moreover, any device with equivalent functions that is produced from a structure formed by a recombination of elements shall fall within the scope of the present invention. Additionally, the drawings are only illustrative and are not drawn to actual size.

The using of “first”, “second”, “third”, etc. in the specification should be understood for identifying units or data described by the same terminology, but are not referred to particular order or sequence.

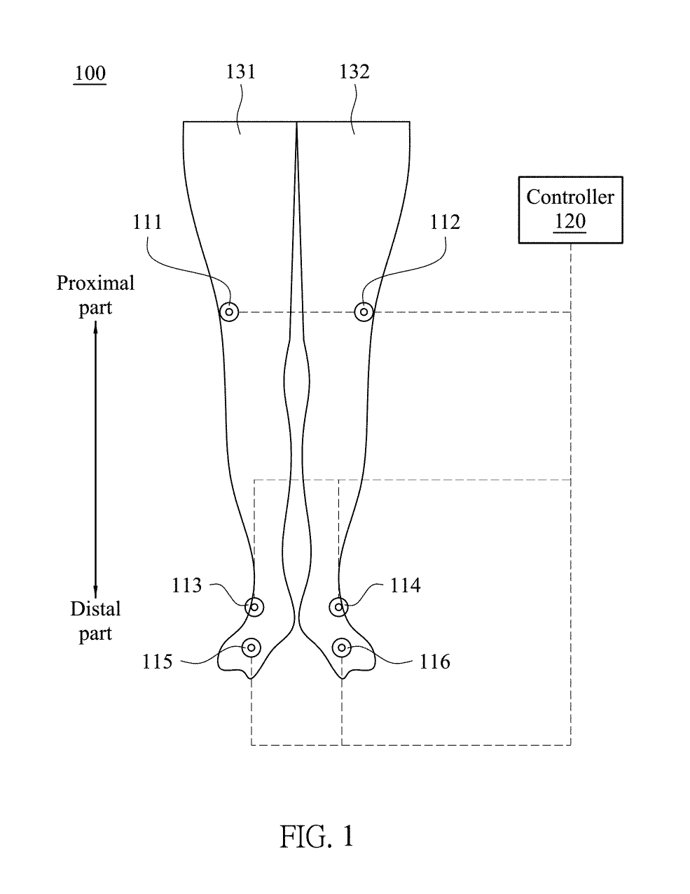

FIG. 1 is a schematic diagram of a gait analyzing system in accordance with an embodiment. Referring to FIG. 1, a gait analyzing system 100 includes multiple accelerometers 111-116 and a controller 120. In some embodiments, the controller 120 is a central processing unit, a microprocessor, a micro-controller, a digital signal processor, a baseband processor, or an application-specific integrated circuit (ASIC). Each of the accelerometers 111-116 has vertical, anterior-posterior, and medio-lateral sensing axes. However, in other embodiments, each of the accelerometers 111-116 may have more or less sensing axes, which is not limited in the invention. The controller 120 receives accelerations sensed on the sensing axes of the accelerometers 111-116 in a wire or wireless way. For example, the controller 120 may obtain the accelerations by wireless fidelity (WiFi), near field communication (NFC), Bluetooth or other suitable communication mechanism.

The accelerometers 111-116 are disposed on two extremities 131 and 132 pair by pair. The accelerometers 111, 113, and 115 on the first lower extremity 131 are paired with the accelerometers 112, 114, and 116 on the second lower extremity 132 respectively. In detail, the accelerometers 111 and 112 are disposed at 3 cm above the lateral epicondyle; the accelerometers 113 and 114 are disposed at 3 cm above the lateral malleolus; and the accelerometers 115 and 116 are disposed on the foot (e.g. 2 cm below the head of the 4^{th} metatarsal). However, the disposition location are just examples, and the accelerometers may be disposed at other suitable locations in other embodiments. The accelerations sensed by each pair of accelerometers (e.g. the accelerometers 111 and 112) can be used to calculate two gait indexes that would be described in detail below.

In some embodiments, after collecting the accelerations sensed by the accelerometers 111-116, the controller 120 may perform some signal pre-processing such as filtering, spatial-to-frequency domain transformation, extreme value removing, etc. For example, the frequency of human gait is generally within a certain bandwidth (e.g. under 15 hertz), and therefore the accelerations may be transformed into the frequency domain by Fourier transform, and then are filtered by a Butterworth filter in which the cut-off frequency is set to be 15 hertz. However, the content of the signal pre-processing is not limited in the invention.

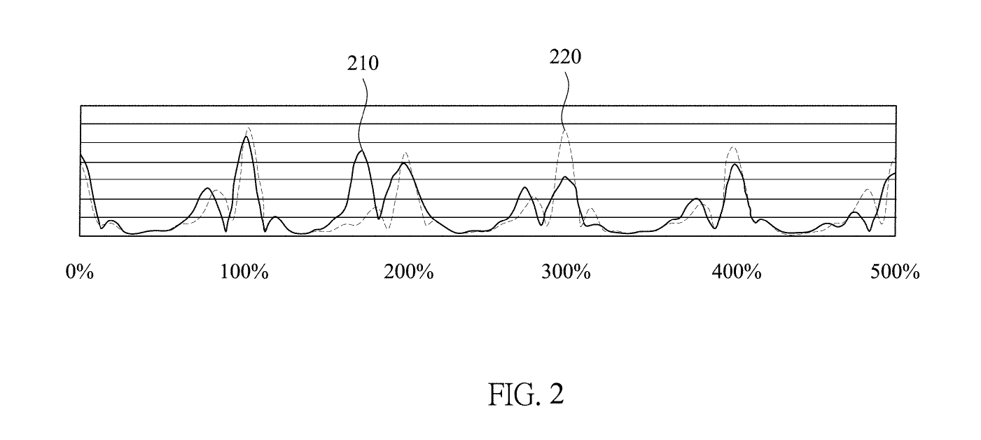

The accelerations are then segmented as multiple strides. FIG. 2 is a schematic diagram of stride segmentation in accordance with an embodiment. Referring to FIG. 1 and FIG. 2, the horizontal axis of FIG. 2 represents time and the vertical axis represents the magnitude of acceleration. Two curves 210 and 220 correspond to the extremities 131 and 132 respectively. One of the lower extremities 131, 132 is set to be a reference lower extremity, and the other one is set to be an opposite lower extremity. The reference extremity is taken as the start of a stride. In some embodiments, the reference extremity is set in accordance with handedness of the user. In some embodiments, the user is a stroke patient who has an affected lower extremity and an unaffected lower extremity, and thus the unaffected lower extremity may be set to be the reference lower extremity. In some embodiments, the user on his own decides which lower extremity to be the reference lower extremity. However, how the reference lower extremity is decided is not limited in the invention. The greatest acceleration along the vertical direction is generated when the heel contacts the ground, and therefore the time between two consequent ground contacts of the reference lower extremity is taken as a stride. People in the art should be able to adopt any suitable stride segmentation algorithm, which is not limited in the invention. In the embodiments, the accelerations of each stride are normalized as N sampling points, and a period of one stride is represented as 100%, in which N is a positive integer such as 120. In FIG. 2, the range from 0% to 500% represents 5 strides. In some embodiments, the user is asked to walk for a distance (e.g. 15 meters) first, and then several middle strides are obtained for the following analysis.

Next, a corresponding root mean square (RMS) value is calculated for each time point (total of N) in one strode and for each accelerometer. To be specific, the accelerations on the three sensing axes are represented as vectors \vec{a_{V}},\vec{a_{AP}},\vec{a_{ML}}, respectively. The RMS value is calculated according to the following equation (1).

$$RSS = \sqrt{{( \vec{{a}_{V}} )}^{2} + {( \vec{{a}_{AP}} )}^{2} + {( \vec{{a}_{ML}} )}^{2}} \qquad ( 1 )$$

Take the accelerometers 115 and 116 for examples, let a1 represent the RMS values of the accelerometer 115, and let a2 represent the RMS values of the accelerometer 116, in which a1, a2 are vectors shown in the following equations (2) and (3).

a1=a1₍₁₎,a1₍₂₎,a1₍₃₎, . . . , a1₍ₙ₎, . . . , a1_{(N)} (2)

a2=a2_{(1),}a2₍₂₎,a2₍₃₎, . . . , a2₍ₙ₎, . . . , a2_{(N)} (3)

a1₍₁₎ represents the RMS value of the accelerations sensed by the accelerometer 115 at the first time point, and so on. Next, a cross correlation coefficient is calculated according to the RMS values of the accelerometers 115 and 116 as the following equation (4).

Cc(k)=Σₙ₌₁^{N}a1₍ₙ₎a2₍ₙ₋ₖ₎ k=0,±1,±2, . . . , ±N−1 (4)
if n−k≤0 or n−k≥N, then a2₍ₙ₋ₖ₎=0

k represents the time point. N is the number of the time points. Cc(k) is the cross correlation coefficient at the time point k. In addition, an auto-correlation coefficient of the RMS values a1, and an auto-correlation coefficient of the RMS values a2 are calculated as the following equations (5) and (6).

Ac1₍ₖ₎=Σₙ₌₁^{N}a1₍ₙ₎a1₍ₙ₋ₖ₎ k=0,±1,±2, . . . , ±N−1 (5)
if n−k≤0 or n−k≥N, then a1₍ₙ₋ₖ₎=0

Ac2₍ₖ₎=Σₙ₌₁^{N}a2₍ₙ₎a2₍ₙ₋ₖ₎ k=0,±1,±2, . . . , ±N−1 (6)
if n−k≤0 or n−k≥N, then a2₍ₙ₋ₖ₎=0

Ac1₍ₖ₎ and Ac2₍ₖ₎ represent the auto-correlation coefficients of the accelerometers 115 and 116 respectively. Then, a first gait index associated with the gait is calculated according to the cross correlation coefficient Cc, the auto-correlation coefficient Ac1 and the auto-correlation coefficient Ac2 as the following equation (7).

$${Cc}_{norm} = \frac{max ( Cc )}{\sqrt{A c {1}_{( 0 )} \times Ac {2}_{( 0 )}}} \qquad ( 7 )$$

max(Cc) represents the maximum value among the cross correlation coefficients Cc(−119) to Cc(119). The first gait index Ccₙₒᵣₘ is in a range from 0 to 1. If the first gait index approximates to 1, it indicates a strong correlation between the RMS values a1 and a2. The greater the first gait index Ccₙₒᵣₘ is, the better the symmetry of the gait is. In some embodiments, the controller 120 can generate a message when the first gait index Ccₙₒᵣₘ is less than a threshold for bad symmetry, or generate a message when the first gait index Ccₙₒᵣₘ is greater than another threshold for good symmetry. The message may be shown to a user in a way of voice, image, text or the like, or may be transmitted to another electrical device, which is not limited in the invention.

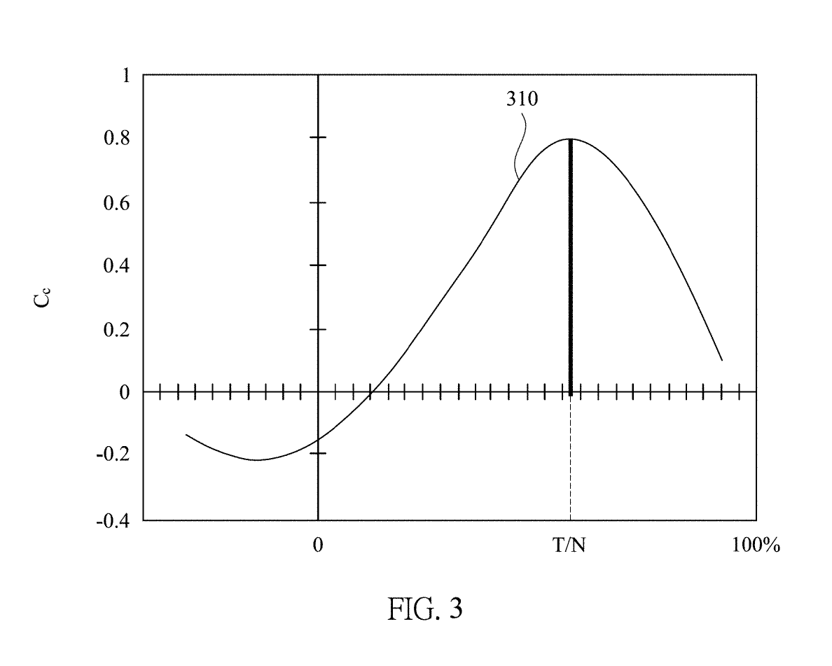

In some embodiments, a second gait index is obtained by calculating a delay time that the cross correlation coefficient Cc reaches its maximum value and by normalizing the delay time by the positive integer N. FIG. 3 is a schematic diagram of calculating the second gait index in accordance with an embodiment. In FIG. 3, the horizontal axis (has been normalized) is time, and the vertical axis is the magnitude of the cross correlation coefficient Cc. A curve 310 reaches its maximum value at delay time T as shown in the following equation (8), in which the delay time T is written as Tmax(Cc). The normalization is performed according to the equation (9).

$$\begin{matrix}T max ( Cc ) = arg {max}_{k} Cc ( k ) & ( 8 ) \\ {T}_{s} = \frac{T max ( Cc )}{N} \times 100 \% & ( 9 )\end{matrix}$$

Note that the sign of the second gait index Ts indicates whether the reference extremity lags behind the opposite extremity or the opposite extremity lags behind the reference extremity. In the embodiment, the less the absolute value of the second gait index Ts is, the more stable the gait is.

One first gait index Ccₙₒᵣₘ and one second gait index Ts are obtained for each stride according to the aforementioned algorithm. In some embodiments, the gait indexes of multiple (e.g. 5) strides are calculated, and the average thereof is outputted. In accordance with the experimental result, stroke fallers have smaller first gait index Ccₙₒᵣₘ and larger second gait index Ts, compared with non-faller. Therefore, the two indexed can be used to identify the stroke falters.

Referring to FIG. 1, the first gait index Ccₙₒᵣₘ and the second gait index Ts are generated for each pair of accelerometers. In some embodiments, the accelerometers 115 and 116 provide richer information because they are located relatively lower, and accordingly, the first gait index Ccₙₒᵣₘ and the second gait index Ts corresponding to the accelerometers 115 and 116 are outputted. However, in some embodiments, the gait indexes corresponding to the accelerometers 111-114 may also be used to analyze the gait, which is not limited in the invention.

In some embodiments, the gait index Ccₙₒᵣₘ and the gait index Ts are used as a portion of a feature vector which is inputted to a machine learning model for determining whether the gait is normal. The machine learning model may be, for example, support vector machine (SVM), neural network or other suitable model. When SVM is adopted, the SVM may be linear or nonlinear. In addition, other parameters may be a portion of the feature vector in addition to the two gait indexes. For example, in some embodiments, a recurrence quantification analysis (RQA) is performed for the RMS values of each accelerometer. Some RQA related measures such as recurrence rate (RR), percent determinism (DET), average length of diagonal line (L), trapping time (TT), recurrence time (RT), and/or entropy (ENT) may be taken as a portion of the feature vector. In some embodiments, the entropy of the RMS values may also be a portion of the feature vector, which is not limited in the invention.

In some embodiments, the feature vector may be generated in accordance with the RMS values of the accelerometers 115 and 116, or generated in accordance with the RMS values of the accelerometers 113-116. In some embodiments, a respective feature vector is generated for each pair of accelerometers 111-116 to determine whether a body part is abnormal. For example, a feature vector is generated in accordance with the RMS values of the accelerometers 115 and 116 for training a first SVM which is used to determine whether the motor control of ankle (e.g. ankle strategy) can compensate the abnormal motor control due to certain conditions; another feature vector is generated in accordance with the RMS values of the accelerometers 113 and 114 for training a second SVM which is used to determine whether the motor control above ankle (e.g. hip strategy) can compensate the abnormal motor control due to certain conditions. Herein, the part above the ankle joint is referred to a proximal part, and the part of the ankle joint and under the ankle joint is referred to a distal part. If determining that the distal part is normal, it means the motor control of the ankle joint is functioning well, and thus the distal part does not need further treatment or training, and whether the proximal part is normal is further determined. If determining that the distal part is abnormal, it means the motor control of the ankle joint is not functioning well, and thus the distal part needs further treatment or training, and whether the proximal part is normal is further determined. If the proximal part is normal, it means the motor control above ankle is functioning well without the need of further treatment or training; if the proximal part is abnormal, it means the motor control above ankle is not functioning well, and thus it needs treatment or training. The treatment or training may be vibration or other suitable treatment or involve some aids, which is not limited in the invention.

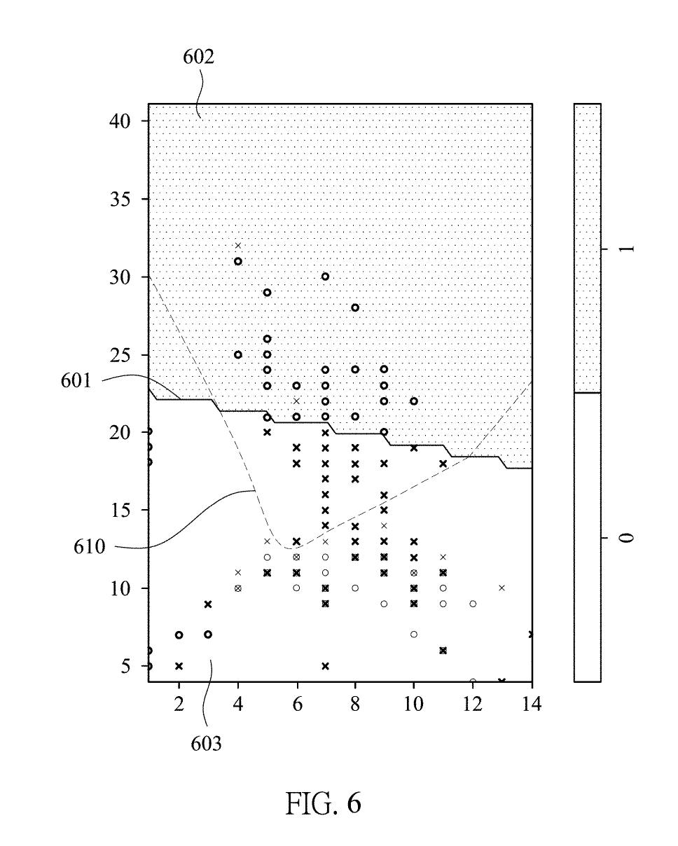

FIG. 6 is a diagram illustrating SVM classification in accordance with an embodiment. Referring to FIG. 6, the horizontal axis and the vertical axis represent two elements in the feature vectors generated in accordance with the RMS values of the accelerometers 115 and 116. Multiple feature vectors are collected, and each feature vector represents a person. The dimension of the feature vectors is higher than two, but only two dimensions are shown in FIG. 6 for simplification. Each feature vector is projected onto the two-dimension diagram as a symbol. The ground truth of each feature vector is either normal person (shown as bold symbol) or stroke patient (shown as non-bold symbol). Each feature vector is classified as either normal person (shown as “o”) or stroke patient (shown as “x”). Therefore, bold “x” indicates false negative, non-bold “o” indicates false positive, and so on. A hyperplane 601 is generated by a linear SVM model to separate the feature space into two areas 602 and 603 in the embodiment of FIG. 6. However, a non-linear SVM model may be applied in another embodiment. For example, a hyperplane 610 may be generated by a non-linear SVM model with radial basis function (RBF) kernel to improve the classification. In addition, the RMS values of the accelerometers 111-114 may be used to illustrate other similar diagrams which are not shown for simplification.

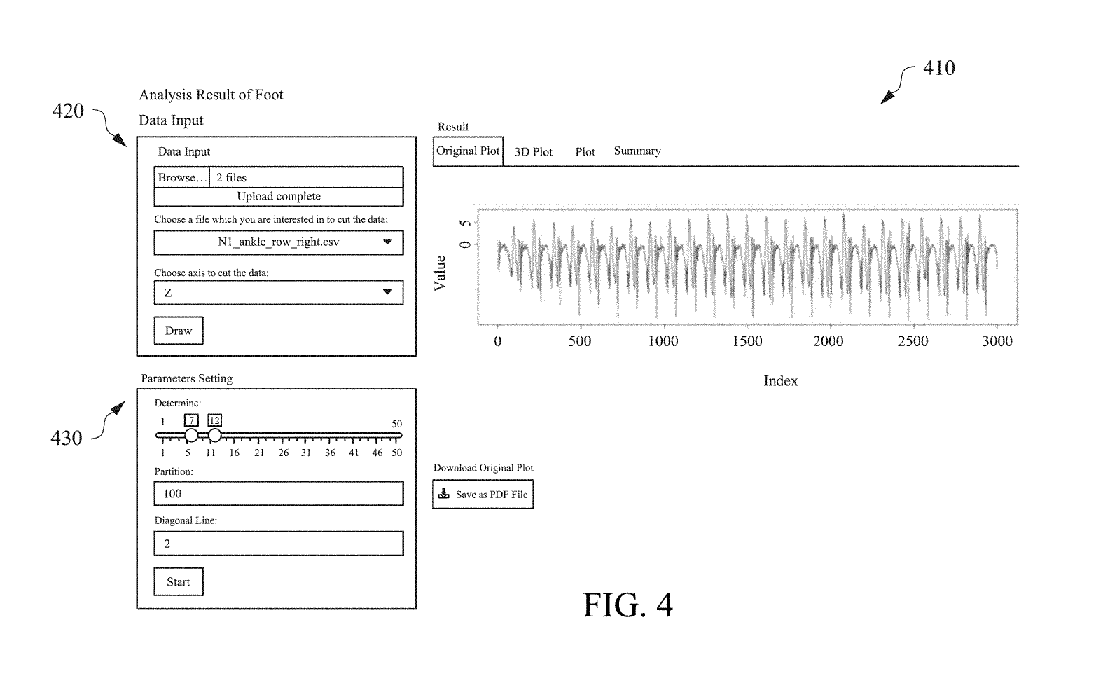

In some embodiments, the controller 120 is electrically connected to or included in an electrical device which may be a personal computer, a server, a smart phone, a tablet or any forms of embedded system. The electrical device also includes a screen on which the controller 120 shows a graphical interface and illustrates related information of the gait analysis on the graphical interface. For example, FIG. 4 is a schematic diagram of the graphical interface in accordance with an embodiment. In the embodiment of FIG. 4, the graphical interface includes a diagram area 410, an input area 420 and a parameter setting area 430. The accelerations, RMS values, recurrence plot, feature vectors, result of the gait analysis, and the like may be shown in the diagram area 410. For example, the RQA is performed on the RMS values of one accelerometer, and the recurrence plot is shown in the diagram area 410. One or multiple graphical objects are provided in the input area 420 for the user to input the file to be analyzed, the sensing axis to be rendered, etc. One or multiple graphical objects are also provided in the parameter setting area 430 for the user to input any kind of parameters. The aforementioned graphical object may be any object with graphical interface such as bottom, menu, text box or other suitable objects. In addition, the graphical interface of FIG. 4 is just an example, how the result of the gait analysis is shown is not limited in the invention.

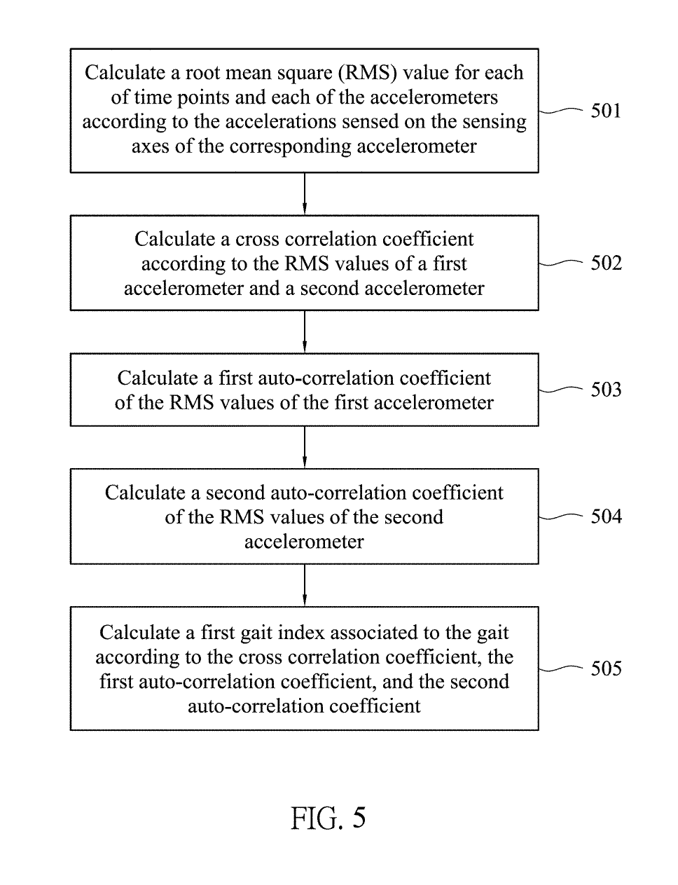

FIG. 5 shows a flow chart of a gait analyzing method in accordance with an embodiment. Referring to FIG. 5, in step 501, a root mean square (RMS) value is calculated for each of time points and each of the accelerometers according to the accelerations sensed on the sensing axes of the corresponding accelerometer. In step 502, a cross correlation coefficient is calculated according to the RMS values of a first accelerometer and a second accelerometer. In step 503, a first auto-correlation coefficient of the RMS values of the first accelerometer is calculated. In step 504, a second auto-correlation coefficient of the RMS values of the second accelerometer is calculated. In step 505, a first gait index associated to the gait is calculated according to the cross correlation coefficient, the first auto-correlation coefficient, and the second auto-correlation coefficient. However, all the steps in FIG. 5 have been described in detail above, and therefore they will not be repeated. Note that the steps in FIG. 5 can be implemented as program codes or circuits, and the disclosure is not limited thereto. In addition, the method in FIG. 5 can be performed with the aforementioned embodiments, or can be performed independently. In other words, other steps may be inserted between the steps of the FIG. 5.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A gait analyzing method for a gait analyzing system comprising a plurality of accelerometers, wherein each of the accelerometers has a plurality of sensing axes, and the gait analyzing method comprises:
calculating a root mean square (RMS) value for each of a plurality of time points and each of the accelerometers according to a plurality of accelerations sensed on the sensing axes of the corresponding accelerometer, wherein the accelerometers comprises a first accelerometer and a second accelerometer, the first accelerometer corresponds to a first lower extremity of a gait, the second accelerometer corresponds to a second lower extremity of the gait, and the first lower extremity is different from the second lower extremity;
calculating a cross correlation coefficient according to the RMS values of the first accelerometer and the second accelerometer;
calculating a first auto-correlation coefficient of the RMS values of the first accelerometer;
calculating a second auto-correlation coefficient of the RMS values of the second accelerometer; and
calculating a first gait index associated to the gait according to the cross correlation coefficient, the first auto-correlation coefficient, and the second auto-correlation coefficient.

2. The gait analyzing method of claim 1, wherein the step of calculating the cross correlation coefficient is performed according to an equation (1):
Cc(k)=Σₙ₌₁^{N}a1₍ₙ₎a2₍ₙ₋ₖ₎ k=0,±1,±2, . . . , ±N−1 (1)
if n−k≤0 or n−k≥N, then a2₍ₙ₋ₖ₎=0
wherein k represents one of the time points, N represents the number of the time points, Cc(k) represents the cross correlation coefficient at the time point k, a1₍ₙ₎ represents the RMS value of the first accelerometer at a time point n, a2₍ₙ₋ₖ₎ represents the RMS value of the second accelerometer at a time point (n−k).

3. The gait analyzing method of claim 2, wherein the step of calculating the first auto-correlation coefficient is performed according to an equation (2):
Ac1₍ₖ₎=Σₙ₌₁^{N}a1₍ₙ₎a1₍ₙ₋ₖ₎ k=0,±1,±2, . . . , ±N−1 (2)
if n−k≤0 or n−k≥N, then a1₍ₙ₋ₖ₎=0
wherein the step of calculating the second auto-correlation coefficient is performed according to an equation (3):
Ac2₍ₖ₎=Σₙ₌₁^{N}a2₍ₙ₎a2₍ₙ₋ₖ₎ k=0,±1,±2, . . . , ±N−1 (3)
if n−k≤0 or n−k≥N, then a2₍ₙ₋ₖ₎=0.

4. The gait analyzing method of claim 3, wherein the step of calculating the first gait index of the gait is performed according to an equation (4):
$${Cc}_{norm} = \frac{max ( Cc )}{\sqrt{A c {1}_{( 0 )} \times Ac {2}_{( 0 )}}} . \qquad ( 4 )$$

5. The gait analyzing method of claim 4, wherein the gait analyzing method further comprises:
calculating a delay time that the cross correlation coefficient reaches a maximum value; and
obtaining a second gait index by normalizing the delay time according to the number of the time points.

6. The gait analyzing method of claim 5, further comprising:
training a machine learning model according to the first gait index and the second gait index, determining whether the gait is normal according to the machine learning model.

7. The gait analyzing method of claim 5, further comprising:
performing a recurrence quantification analysis on the RMS values of one of the accelerometers, and displaying a recurrence plot on a screen.

8. A gait analyzing system comprising:
a plurality of accelerometer, wherein each of the accelerometers has a plurality of sensing axes, the accelerometers comprises a first accelerometer and a second accelerometer, the first accelerometer corresponds to a first lower extremity of a gait, the second accelerometer corresponds to a second lower extremity of the gait, and the first lower extremity is different from the second lower extremity; and
a controller, configured to receive a plurality of acceleration sensed on the sensing axes of each of the accelerometers,
wherein the controller calculates a root mean square (RMS) value for each of a plurality of time points and each of the accelerometers according to the accelerations sensed on the sensing axes of the corresponding accelerometer, calculates a cross correlation coefficient according to the RMS values of the first accelerometer and the second accelerometer, calculates a first auto-correlation coefficient of the RMS values of the first accelerometer, calculates a second auto-correlation coefficient of the RMS values of the second accelerometer, and calculates a first gait index associated to the gait according to the cross correlation coefficient, the first auto-correlation coefficient, and the second auto-correlation coefficient.

9. The gait analyzing system of claim 8, wherein the controller calculates the cross correlation coefficient according to an equation (1):
Cc(k)=Σₙ₌₁^{N}a1₍ₙ₎a2₍ₙ₋ₖ₎ k=0,±1,±2, . . . , ±N−1 (1)
if n−k≤0 or n−k≥N, then a2₍ₙ₋ₖ₎=0
wherein k represents one of the time points, N represents the number of the time points, Cc(k) represents the cross correlation coefficient at the time point k, a1₍ₙ₎ represents the RMS value of the first accelerometer at a time point n, a2₍ₙ₋ₖ₎ represents the RMS value of the second accelerometer at a time point (n−k).

10. The gait analyzing system of claim 9, wherein the controller calculates the first auto-correlation coefficient according to an equation (2):
Ac1₍ₖ₎=Σₙ₌₁^{N}a1₍ₙ₎a1₍ₙ₋ₖ₎ k=0,±1,±2, . . . , ±N−1 (2)
if n−k≤0 or n−k≥N, then a1₍ₙ₋ₖ₎=0
wherein the controller calculates the second auto-correlation coefficient according to an equation (3):
Ac2₍ₖ₎=Σₙ₌₁^{N}a2₍ₙ₎a2₍ₙ₋ₖ₎ k=0,±1,±2, . . . , ±N−1 (3)
if n−k≤0 or n−k≥N, then a2₍ₙ₋ₖ₎=0.

11. The gait analyzing system of claim 10, wherein the controller calculates the first gait index according to an equation (4):
$${Cc}_{norm} = \frac{max ( Cc )}{\sqrt{A c {1}_{( 0 )} \times Ac {2}_{( 0 )}}} . \qquad ( 4 )$$

12. The gait analyzing system of claim 11, wherein the controller calculates a delay time that the cross correlation coefficient reaches a maximum value, and obtains a second gait index by normalizing the delay time according to the number of the time points.
